(19) United States
(12) Patent Application Publication
(10) Pub. No.: US 2015/0004143 A1
(43) Pub. Date: Jan. 1, 2015

(54) Use of a Proteolytic Enzyme

(71) Applicant: Glykos Finland Oy, Helsinki (FI)

(72) Inventors: Erja Kerkela, Helsinki (FI); Tanja Hakkarainen, Helsinki (FI); Johanna Nystedt, Helsinki (FI); Jukka Partanen, Helsinki (FI)

(21) Appl. No.: 14/349,802

(22) PCT Filed: Oct. 4, 2012
(86) PCT No.: PCT/FI2012/050952

§ 371 Date: Apr. 4, 2014

(30) Foreign Application Priority Data
Oct. 5, 2011 (FI) 20115976

(51) Int. Cl. A61K 35/28

(52) U.S. Cl. CPC A61K 35/28
USPC 424/93.7; 435/375

(57) ABSTRACT

The present invention relates to a hematopoietic stem cell and/or a population thereof having a specific profile of cell surface proteins and/or proteoglycans. The present invention also relates to use of proteolytic enzymes, such as pronase and pronase-like enzymes in the modification of the cell surface of a hematopoietic stem cell. The present invention further relates to a method of modifying the cell surface of a hematopoietic stem cell by treatment with proteolytic enzymes, such as pronase and pronase-like enzymes.

### FIELD OF THE INVENTION

The present invention relates to a hematopoietic stem cell and/or a population thereof having a specific profile of cell surface proteins and/or proteoglycans. The present invention also relates to use of a proteolytic enzyme in the modification of the cell surface of a hematopoietic stem cell. The present invention further relates to a method of modifying the cell surface of a hematopoietic stem cell by treatment with a proteolytic enzyme.

### BACKGROUND OF THE INVENTION

Proteolytic enzymes are a large group of enzymes which are involved in digesting protein chains into shorter fragments by splitting the peptide bonds that link amino acid residues together. Some of them are able to detach the terminal amino acids from the peptide or protein chain (exopeptidases, such as aminopeptidases) and the others attack internal peptide bonds of a protein (endopeptidases, such as trypsin, chymotrypsin, pepsin, papain, elastase). Proteolytic enzymes can be divided into four major groups according to the character of their catalytic active site and conditions of action: serine proteinases, cysteine (thiol) proteinases, aspartic proteinases, and metalloproteinases. Classification of a protease to a certain group depends on the structure of catalytic site and the amino acid (as one of the constituents) essential for its activity.

Pronase is a proteolytically non-specific, commercially available mixture of proteinases isolated from the extracellular fluid of Streptomyces griseus. Its proteolytic activity is attributable to the composition of the preparation, which comprises various types of endopeptidase (serine and metalloproteases) and exopeptidase (carboxypeptidases and aminopeptidases). Typically, neutral protease, chymotrypsin, trypsin, carboxypeptidase, and aminopeptidase are present, together with neutral and alkaline phosphatases. The preparation is, however, free from nucleases.

Stem cells are characterized by their ability to renew themselves through mitotic cell division and to differentiate into a diverse range of cell types. The two main types of mammalian stem cells are embryonic stem cells and adult stem cells, such as hematopoietic stem cells, mesenchymal stem cells, endothelial stem cells and tissue-specific stem cells. Induced pluripotent stem (iPS) cells are derived from adult tissues but converted to embryonic stem cell like cells.

Hematopoietic stem cells (HSC) form progenitors for practically all cell types found in the blood. HSC are currently used for treating many malignant hematological diseases, in particular leukemias and also certain nonhematological diseases. HSCs can be found in and typically are isolated from, for example, bone marrow and cord blood. Usually, HSC are selected using CD34 or CD133 as markers; but similar to other stem cells there are no definitive cell surface markers for HSC (e.g. Spangrude, Uchida and Weissman: Hematopoietic stem cells: biological targets and therapeutic tools. In Atkinson et al, eds: Clinical Bone Marrow and Blood Stem Cell Transplantation, pp 13-37. Cambridge Univ Press, Cambridge U.K., 3^{rd} ed, 2004). HSC can also be isolated from peripheral blood.

A problem related to hematopoietic stem cell transplantation as done using current standards is entrapment (may also be called as “distribution” or “homing”) of the transplanted cells to unwanted organs or tissues. The term “homing” here refers to targeted trafficking of cells to certain tissues or organs; often mediated by specific cell surface molecules and soluble chemokines. One example of undesired distribution of cells is the observed phenomena of lung entrapment: intravenously infused MSCs are rapidly trapped in the lungs in animal models (Gao et al. 2001. The dynamic in vivo distribution of bone marrow-derived mesenchymal stem cells after infusion. Cells Tissues Organs 169, 12-20; Schrepfer et al. 2007. Stem cell transplantation: the lung barrier. Transplant Proc 39: 573-576). Lung entrapment is not limited to MSCs, since trapping of cells in the lungs occurs also with metastatic tumors (Khanna et al 2004. The membrane-cytoskeleton linker ezrin is necessary for osteosarcoma metastasis. Nature Med 10, 182-186) and with HSCs during acute distribution (Kang et al. 2006: Tissue distribution of 18F-FDG-labeled peripheral hematopoietic stem cells after intracoronary administration in patients with myocardial infarction. J Nucl Med. 47:1295-301).

An approach to increase the portion of the cells that find their way to the intended target organ or tissue, has been increasing the number of cells in a graft. However, by this approach the increased dose unfortunately tends to result in higher rates of clinical complications, for example, graft-versus-host disease, a possible fatal condition after HSC transplantation. Also, it is sometimes not feasible to get a higher number of cells for transplantation, for example, a single unit of cord blood, a suitable source for HSC, has a limited number of stem cells. Expansion ex vivo provides one option to get a higher number of stem cells but it is currently not established that the expanded cells have the same properties as the original cells. Hence, a more efficient use of stem cells of a graft is warranted.

It has now been discovered that by processing and/or treating hematopoietic stem cells with pronase enzyme, a characteristic cell surface profile is achieved in which certain proteins are missing whereas the others remaining intact. As proteins cut off from the cell surface by pronase act in targeting of the cells in tissues not desired in HSC therapy, the treatment changes their biological distribution and properties, hence resulting in more efficient therapeutic products. The targeting of the cells is changed so that they should not entrap into the lung but are more efficiently available in the desired tissue.

### BRIEF DESCRIPTION OF THE INVENTION

An object of the present invention is to provide a hematopoietic stem cell (HSC) and/or a population thereof, having a specific profile of cell surface proteins and/or proteolglycans. Another object of the present invention is to provide a method of modifying the cell surface of a HSC by treatment with a proteolytic enzyme, such as pronase or a pronase-like enzyme. Another object of the invention is a use of pronase and/or a pronase-like enzyme in the modification of the cell surface of a therapeutic HSC preparation.

In particular, an object of the present invention is to provide a method of assisting transplanted hematopoietic stem cells to the target organ(s). Another object of the present invention is to provide a method of hindering and/or preventing the transition of hematopoietic stem cells from blood stream to organs which are not the actual target ones, e.g., the lungs and/or liver. A further object of the present invention is to provide a method of modifying and/or altering the distribution behaviour of cells used for cellular therapy.

The invention is based on the observation that HSC treated with pronase are entrapped to a lesser extent to the lungs, i.e., to organs which are not the actual targets of the stem cell graft, than HSC that have not been treated with pronase or have been treated with trypsin, another proteolytic enzyme.

Accordingly, the present invention provides a novel and effective means for assisting the transition of HSC of a graft from blood stream to the target organ(s) and optionally simultaneously hindering and/or preventing the transition of HSC of a graft from blood to organs which are not the actual targets, i.e., the lungs and/or liver. In addition, the present invention provides a novel and effective means for modifying and/or altering the “homing” properties or behaviour of the cells.

The objects of the invention are achieved by the methods, uses and cells and/or cell populations set forth in the independent claims. Preferred embodiments of the invention are described in the dependent claims.

Other objects, details and advantages of the present invention will become apparent from the following drawings, detailed description and examples.

### BRIEF DESCRIPTION OF THE DRAWINGS

FIG. 1 lists the antibodies used in the invention.

FIG. 2 shows the expression of selected cell surface proteins after 0.5% pronase treatment (“pronase cells”) of UCB CD34-enriched cells and in the control cells (untreated). Results are presented as the percentage of positive cells as studied by flow cytometry, averages from different experiments when possible. stdev=standard deviation, n refers to the number of experiments.

FIG. 3 shows the detailed images from the cell surface profiling by flow cytometry from UCB CD34-enriched cells, both pronase-treated and untreated, analysed with FACSdiva software (BD).

FIG. 4 shows the expression of selected cell surface proteins in UCB MNC in controls and after 0.5% pronase treatment. Results are presented as the percentage of positive cells.

FIG. 5 shows the detailed images from the cell surface profiling by flow cytometry from UCB MNC, both pronase-treated and untreated, analysed with FACSdiva software (BD).

FIG. 6 shows the recovery and viability of UCB MNC when treated with 0.5% pronase in buffer versus medium as well as corresponding controls.

FIG. 7 shows the results from the optimisation experiment of pronase treatment of MNC. The effect of different pronase concentrations and incubation times was studied with the cell surface expression of CD44, CD162 and CD54 by flow cytometry. Results are presented as percentage of positive cells. NA=not analysed.

FIG. 8 shows the results of CFU assay for control (untreated) and pronase-treated UCB CD34+ cells. tot=total.

FIG. 9 shows the amounts and viabilities for control (untreated) mouse cells, after the pronase treatment as well as after overnight (O/N) incubation. NA=not analysed.

FIG. 10 shows the expression of selected cell surface proteins in mouse cells in controls and after 0.5% pronase treatment as well as after overnight (O/N) incubation. Results are shown as percentage of positive cells. Both c-kit-enriched and whole BM cells were studied. Note that CD54 and CD49d were both studied only once.

FIG. 11 shows the detailed images from the cell surface profiling by flow cytometry from mouse c-kit enriched cells, both pronase-treated and untreated as well as after overnight (O/N) incubation following the pronase treatment, analysed with FACSdiva software (BD).

FIG. 12 shows the results of CFU assay for control (untreated) and pronase-treated mouse cells. tot=total.

### DETAILED DESCRIPTION OF THE INVENTION

The present invention is based on the finding that treatment with pronase produces characteristic changes in the protein epitope profile of the hematopoietic stem cell (HSC) and the human umbilical cord blood mono-nuclear cell (UCB MNC) surfaces as demonstrated by the disappearance of certain antibody-binding epitopes.

In particular, the bindings of the anti CD44, anti CD49e and anti CD162 antibodies are practically completely vanished by treating HSC with pronase, the staining being about 1%-4%, that is, practically 0%, while 80%-95% of the untreated HSC express them. The result indicates a cleavage of the hyaluronan receptor CD44 (UniProt id P16070), integrin CD49e (α5; UniProt id P08648) and P-selectin glycoprotein ligand-1 CD162 (UniProt id Q14242) from the cell surface of HSC by pronase. All these molecules are known to have many functions related to cell, including stem cell targeting and homing. CD44 and CD49e also are known to bind to fibronectin, a component possibly targeting cells to the lung. The present invention also demonstrates that HSC indeed bind to fibronectin.

In addition, a clear reduction of CD54 (ICAM-1; UniProt id P05362) and CD55 (Decay accelarating factor DAF; UniProt id P08174) epitopes can be seen, from 85% -95% in untreated HSC to 13% -17% in pronase-treated HSC.

Certain proteins remained intact on the surface of HSC after the pronase treatment: no differences were detected by anti CD34 (function not established; UniProt id P28906) and anti CD133 (Prominin-1, function not established; UniProt id O43490) antibodies, hence, the HSC markers remained intact, nor by anti CD49d (integrin α4; UniProt id P13612) or anti CD45 (protein tyrosine phosphatase receptor; UniProt id P08575) antibodies.

Essentially similar results were obtained using human UCB MNC and mouse HSC.

In one embodiment, the present invention provides a hematopoietic stem cell and/or a population thereof having cell surface protein profile wherein proteins CD44, CD49e and CD162 are “essentially missing”, that is less than 20%, preferably less than 10% or even more preferentially less than 4% of the cells are positive for the marker. In another embodiment, any one of proteins CD44, CD49e or CD162 is “essentially missing”. In a further embodiment any combination of two proteins from the three, that is, CD44 and CD49e, or CD44 and CD162, or CD49e and CD162 is “essentially missing”.

In another embodiment of the invention, the level of at least one of the proteins CD54 or CD55 is additionally “diminished”, that is, less than 30%, preferably less than 20% of the cells are positive for the marker.

Furthermore in one embodiment, the present invention provides a hematopoietic stem cell and/or a population thereof having the cell surface protein profile wherein proteins CD44, CD49e and CD162 are “essentially missing” and at least one of proteins CD49d, CD34, CD133 or CD45 are “present” in the profile, that is, their expression levels are essentially not changed from those observed in untreated or control cells, or generally detected in cells that contain these proteins on their surface. In another embodiment, the present invention provides a hematopoietic stem cell and/or a population thereof having the cell surface protein profile wherein at least one of the proteins CD44, CD49e or CD162 is “essentially missing” and at least one of proteins CD49d, CD34, CD133 or CD45 is “present” in the profile. In another embodiment, the present invention provides a hematopoietic stem cell and/or a population thereof having the cell surface protein profile wherein proteins CD44, CD49e and CD162 are “essentially missing” and all the proteins CD49d, CD34, CD133 and CD45 are “present” in the profile.

In a further embodiment, the present invention provides a stem cell and/or a population thereof having the cell surface protein profile, wherein

(i) proteins CD44, CD49e and CD162 are “essentially missing”, and/or

(ii) at least one of proteins CD54 or CD55 is diminished, and/or

(iii) proteins CD49d, CD34, CD133 or CD45 are “present” in the profile.

In a further embodiment, the present invention provides a stem cell and/or a population thereof having the cell surface protein profile, wherein

(i) proteins CD44, CD49e and CD162 are “essentially missing”, and

(ii) at least one of the proteins CD54 or CD55 is diminished, and

(iii) proteins CD49d, CD34, CD133 or CD45 are “present” in the profile.

In an even further embodiment, the present invention provides a stem cell and/or a population thereof having the cell surface protein profile, wherein

(i) at least one of the proteins CD44, CD49e and CD162 is “essentially missing”, and/or

(ii) at least one of the proteins CD54 or CD55 is diminished, and/or

(iii) at least one of the proteins CD49d, CD34, CD133 or CD45 is “present” in the profile.

In one embodiment, in addition to at least one of proteins CD49d, CD34, CD133 and/or CD45, at least one of proteins CD29, CD18, CD11a, and/or CD184 are found in the cell surface profile of the cell population at the similar levels as in untreated cells. In another embodiment, in addition to proteins CD49d, CD34, CD133 and CD45, proteins CD29, CD18, CD11a and CD184 are found in the cell surface profile of the cell population at the similar levels as in untreated cells.

A hematopoietic stem cell and/or a population thereof having one of the above characterized cell surface protein profiles can be produced by treating the cell or the population thereof with pronase or by preventing the expression of genes coding these molecules by a specific inhibitor and/or by any suitable gene technological means.

In the present invention the term “hematopoietic stem cell” refers to a hematopoietic stem (HSC) or to a human umbilical cord blood mononuclear cell (UCB MNC). In one embodiment, the cell population treated is a hematopoietic stem population. In another embodiment, the cell population treated is a human umbilical cord blood mononuclear cell population.

Here term “essentially missing” refers to a level in which less than 20%, preferably less than 10%, even more preferably less than 4% of cells are positive for the marker. In one embodiment, term “essentially missing” refers to a level in which less than 10% of cells are positive for the marker. In another embodiment, term “essentially missing” refers to a level in which less than 4% of cells are positive for the marker. Term “diminished” here refers to a level in which less than 30%, preferably less than 20%, and even more preferably less than 15% of the cells are positive for the marker. In one embodiment, term “diminished” refers to a level in which less than 20% of the cells are positive for the marker. In another embodiment, term “diminished” refers to a level in which less than 15% of the cells are positive for the marker. Term “present” in the profiles refers to the essentially equal levels to those found in untreated or control cells or generally detected in cells that contain these proteins in their surface; preferably more than 80%, or more preferably more than 90% of the cells being positive. The detection or determination of the expression level can be done in various methods known in the art, for example, it can be based on antibody epitopes or mass spectrometric analysis. Further, it is generally known that depending on the exact conditions of enzymatic treatment e.g. time, concentration of the enzyme, buffer and/or temperature, the level of decrease of the proteins on the cell surface can vary.

The hematopoietic stem cells and/or the populations thereof having the cell surface protein profile according to the present invention are suitable to be used as a clinical graft for transplantation or in cellular therapy. They are found to a lesser extent to entrap or “home” to organs such as, the lungs and liver, which are not the actual target organs of the stem cell graft.

The results indicate that a different cell surface can be produced by pronase and/or pronase-like enzyme treatment and that for many cell surface antigens the effect is transient with a recovery process initiated after a certain time period, such as within a few hours or overnight. The altered cell surface is transient, implying that the cells gain back their original cell surface profile, apparently with their functional properties as well. The recovery, however, is not too rapid for effective changes in tissue targeting of the cells or for its effective application in the clinical setting. Pronase treatment does not, however, affect in vitro multipotency capacity of the stem cells: pronase treatment did not change the CFU results. This implies that the treatment with pronase does not destroy the therapeutic potential of HSC cells.

The treatment of certain stem cells by proteases, typically trypsin, for detachment is described in prior art but what was surprising in the present invention was the particular cell surface profile and the “homing” properties of the treated hematopoietic stem cells. Thus, the invention is based on the finding that stem cells treated with the proteolytic enzyme, pronase, are to a lesser extent entrapping or “homing” to the lungs and liver, i.e., to organs which are not the actual targets of the hematopoietic stem cell graft in e.g. typical HSC transplantation, than hematopoietic stem cells that have not been treated or have been treated with trypsin.

On the basis of this finding, a method of modifying the cell surface of HSC by contacting it with pronase or a pronase-like enzyme has been developed. The hematopoietic stem cell and/or a population thereof treated with pronase or a pronase-like enzyme have a unique cell surface protein and/or proteoglycan profile. Further, a smaller number of the pronase-treated cells find their way to the organs which are not their actual targets.

Accordingly, the present invention provides a novel and effective means for assisting the transition of transplanted hematopoietic stem cells to the target organ(s) and optionally simultaneously hindering and/or preventing the transition of the cells from the blood to organs which are not their actual targets, i.e., the lungs and/or liver. In addition, the present invention provides a novel and effective means for modifying and/or altering the distribution properties and behaviour of therapeutic cells.

The HSC treated with pronase or a pronase-like enzyme remain stem cell-like and maintain the characteristics typical and peculiar to stem cells.

In the present invention, the term “proteolytic enzyme” refers to pronase and “a pronase-like enzyme”. In the present invention, the term “pronase-like enzyme” refers to a proteolytic enzyme or a mixture of enzymes that cleavages proteins and/or peptide chains essentially similarly than pronase or has an essentially similar mixture of enzymes as found in typical pronase preparations, such as metalloendopeptidase, aminopeptidase, trypsin, putative secreted subtilisin-like serine protease, carboxypeptidase, chain E of structures of product and inhibitor complexes of Streptomyces Griseus protease A, and aminopeptidase S. In one embodiment, the pronase-like enzyme refers to at least one of the enzymes selected from the list containing: metal-loendopeptidase, aminopeptidase, putative secreted subtilisin-like serine protease, carboxypeptidase, chain E of structures of product and inhibitor complexes of Streptomyces Griseus protease A, and aminopeptidase S.

In the present invention, the term “proteolytic enzyme” does not refer to trypsin. In one embodiment of the present invention, the proteolytic enzyme is pronase. In one embodiment of the present invention, the pronase comprises neutral protease, chymotrypsin, trypsin, carboxypeptidase, and aminopeptidase together with neutral and alkaline phosphatases, but is free from nucleases. In another embodiment, the pronase comprises metalloendo-peptidase, aminopeptidase, trypsin, putative secreted subtilisin-like serine protease, carboxypeptidase, chain E of structures of product and inhibitor complexes of Streptomyces Griseus protease A, and aminopeptidase S.

Pronase treatment can optionally be combined with other treatments and/or modifications, such as trypsin treatment before the pronase treatment. Further, the pronase treatment can be combined with other suitable treatments, such as enzymatic modification of glycan structures of glycoproteins. Examples of this are addition of fucose by fucosyl transferases, or addition or removal of sialic acid residues (WO 2008 087256; Xia et al. 2004. Surface fucosylation of human cord blood cells augments binding to P-selectin and E-selectin and enhances engraftment in bone marrow. Blood 104:3091-3096; Sackstein et al. 2008. Ex vivo glycan engineering of CD44 programs human multipotent mesenchymal stromal cell trafficking to bone. Nature Med 14:181-187).

Accordingly, the present invention relates to use of pronase or a pronase-like enzyme for the modification of the cell surface of a therapeutic HSC and/or UCB MNC. The present invention relates also to a method of modifying the cell surface of a HSC and/or UCB MNC by treating the cell with pronase or pronase-like enzyme. In one embodiment of the invention, the cell is a hematopoietic stem cell and/or a population thereof. In another embodiment of the invention, the cell is human umbilical cord blood mononuclear cell or a population thereof. The method may also contain additional and/or optional steps that are conventional to methods of modifying cells, such as washing, incubating and dividing the cell populations.

In a typical embodiment, just to give an overview, HSC cells are contacted with 0.1%-1% (w/V) pronase in standard PBS buffer, pH 7.2,supplemented with EDTA for 1 to 5 minutes or longer if needed. Pronase can be obtained e.g. from Roche (#10165921001 or equivalent). The pronase can be in a soluble form or it may have been attached to a solid phase using various linking techniques well known in the art. The enzyme linked to a solid phase can be readily removed from the cells, hence diminishing possible contaminants not accepted in therapeutic products. After the treatment the cells are pelleted by centrifugation, washed in an appropriate buffer and resuspended in a buffer depending on the intended use of the cells. The cell number and viability can be determined using standard assays. The cells can be applied as fresh or stored in a freezer in a suitable storage buffer.

The transition of hematopoietic stem cells of a graft to the target organ of an individual was found to be enhanced when the cells were treated with pronase before administering to an individual or to the blood stream of an individual in the need of such engraftment. In addition, the transition of hematopoietic stem cells of a graft from blood stream to an organ that is not the actual target organ, such as lungs and liver, was found to be decreased when the cells were treated with pronase before administering to an individual in the need of such engraftment.

Accordingly, the present invention additionally relates to a method of assisting the transition of hematopoietic stem cells of a graft to the target organ of an individual by treating the cells with pronase or a pronase-like enzyme, and injecting them to the individual in the need of such engraftment. Further, the present invention relates to a method of assisting the transition of HSC of a graft from the blood stream to the target organ of an individual by treating the cells with pronase or a pronase-like enzyme and injecting them to the blood stream of an individual in the need of such engraftment. In one embodiment of the invention, the cells are treated with the proteolytic enzyme, such as pronase, in vitro. Furthermore, the present invention relates to a method of hindering and/or preventing the transition of hematopoietic stem cells of a graft from blood stream to an organ that is not the actual target one by treating the cells with pronase or a pronase-like enzyme. In one embodiment of the invention, the organ that is not the actual target organ is lungs and/or liver.

The present invention also relates to a method of modifying and/or altering the distribution behaviour of hematopoietic stem cells in a graft with the treatment of pronase or a pronase-like enzyme. In one embodiment, the treatment is performed in vitro.

The present invention further relates to a hematopoietic stem cell and/or a population thereof having cell surface protein and/or proteoglycan profile resulting from the treatment with pronase or a pronase-like enzyme.

It is of note that in addition to the stem cells, many other cells types are known for cellular therapy. Many of them have the kind of problems related to trapping to unwanted tissues. Other cell types include regulatory T lymphocytes and macrophages that are used for immunomodulatory effects, and cytotoxic T lymphocytes and natural killer (NK) cells used for targeted immune response. In all these cases the therapeutic efficiency of the preparation can be augmented by hindering entrapping in the lung or liver. These cells can be applied alone or together with some other cells, such as stem cells.

It will be obvious to a person skilled in the art that, as the technology advances, the inventive concept can be implemented in various ways. The invention and its embodiments are not limited to the examples described above but may vary within the scope of the claims.

The invention will be described in more detail by means of the following examples. The examples are not to be construed to limit the claims in any manner whatsoever.

### EXAMPLES

### Example 1

### Changes of Cell Surface Epitopes in Hematopoietic Stem Cells After Pronase Treatment

### Materials and Methods

The isolation of human umbical cord blood (UCB)-derived mononuclear cells (MNCs) was done with Ficoll-Hypaque density gradient (Amersham Biosciences, Piscataway, N.J.) according to the manufacturer's instructions. After the isolation, CD34 positive cells were enriched using a single-column separation kit (Direct CD34 Progenitor Cell Isolation-kit, #130-046-702, Miltenyi Biotec), according to the manufacturer's instructions.

Pronase treatment: The cells were treated in 2 mL of 0.5% (w/V) pronase (Roche, #10165921001) in PBS without Ca & Mg, pH 7.2, supplemented with 0.25 mM EDTA. The cells were pelleted by centrifugation (300×g, 5 min) and resuspended in PBS without Ca & Mg, pH 7.2+0.3% human serum album (HSA, Albuman 200 g/L, Sanquin) or bovine serum albumin (BSA, ultrapure, Sigma). Cell number and viability were determined for all samples after every test by nucleocounter (Chemometec) or Trypan blue exclusion.

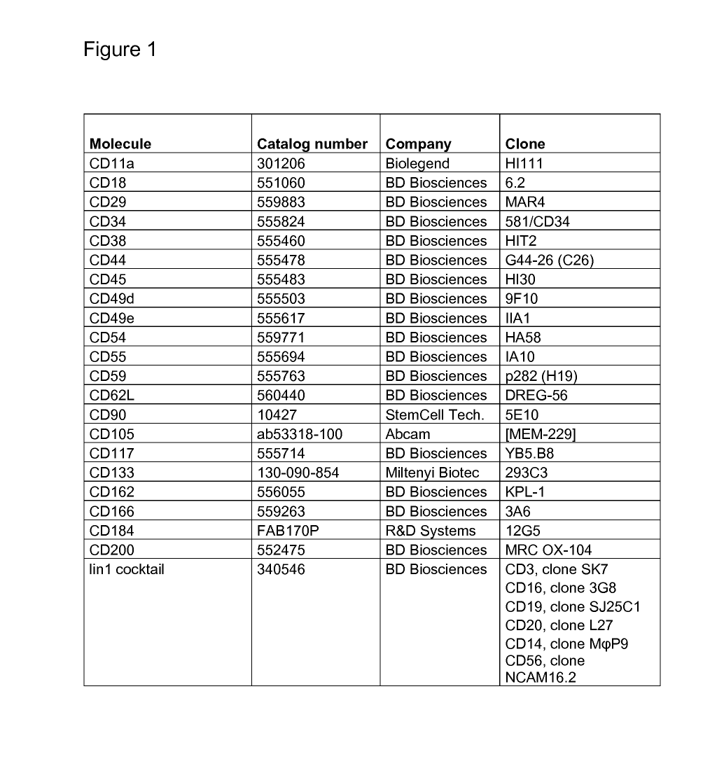

Flow cytometry: To check the expression of cell surface proteins or their antibody epitopes after the pronase treatment, the cells were labelled for flow cytometry with antibodies against several hematopoietic markers and other cell surface molecules, including CD34 (all from BD Biosciences unless stated otherwise), CD90, CD117, CD133 (Miltenyi, 130-090-854), CD38, lin1 cocktail (CD3, 14, 16, 19, 20, 56, BD cat. no. 340546), CD11a, CD29, CD44, CD49d (VLA-4 or ITG α4), CD49e, CD54, CD55, CD59, CD200, CD184 (R&D systems), CD162, CD62L. All the antibodies used are listed in FIG. 1. Untreated cells were used as control. The cells were labelled with 2 μL of antibody per 5×10e4-1×10e5 cells in PBS pH 7.2+0.3% human serum albumin (HSA) or bovine serum albumin (BSA) for 30 minutes on ice. After washing with excess labelling buffer, the labelled cells were run with a FACSAria (BD) flow cytometer and the results were analyzed with the FACSDiva software (BD).

### Results

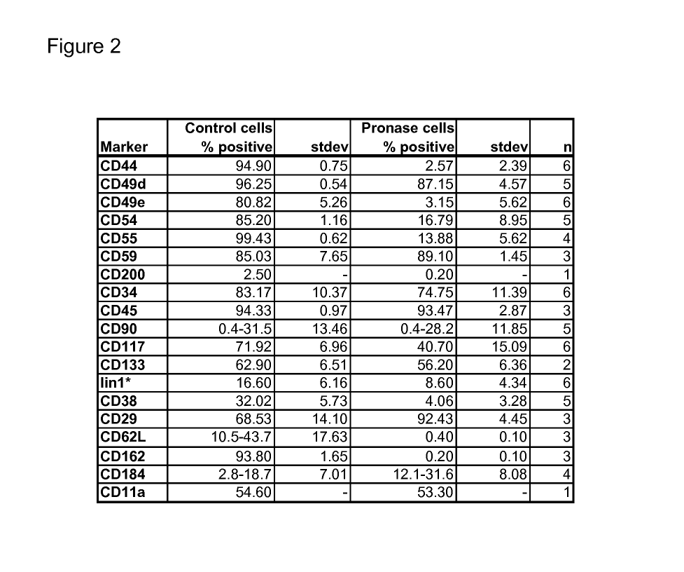

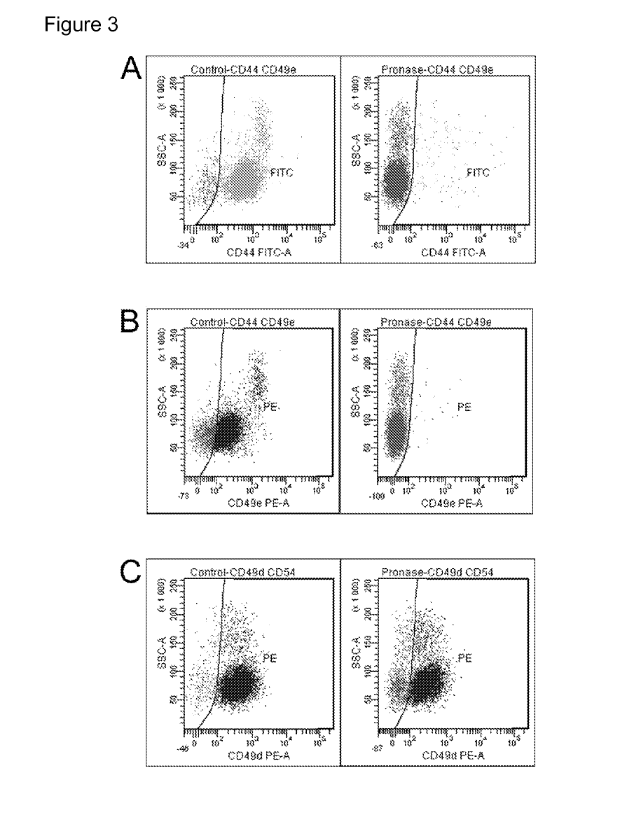

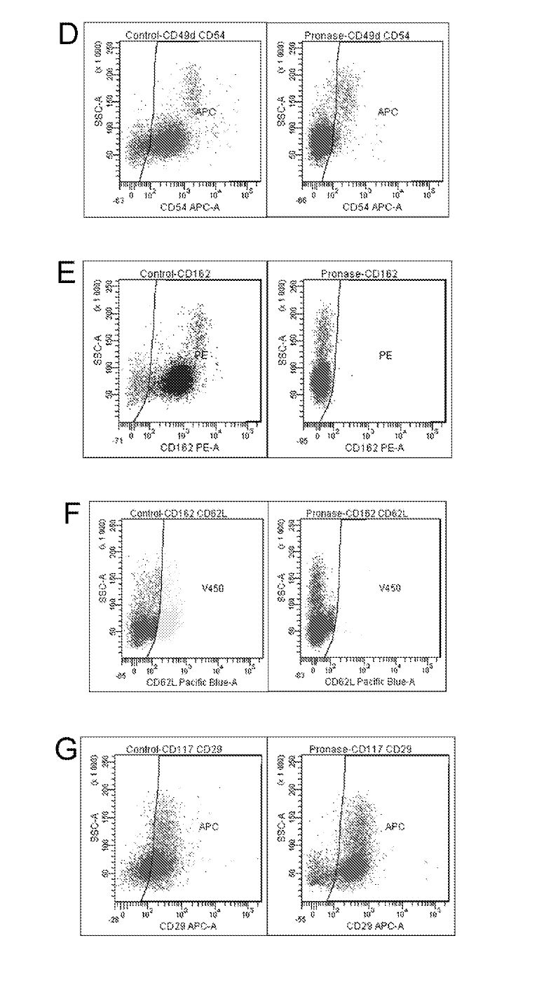

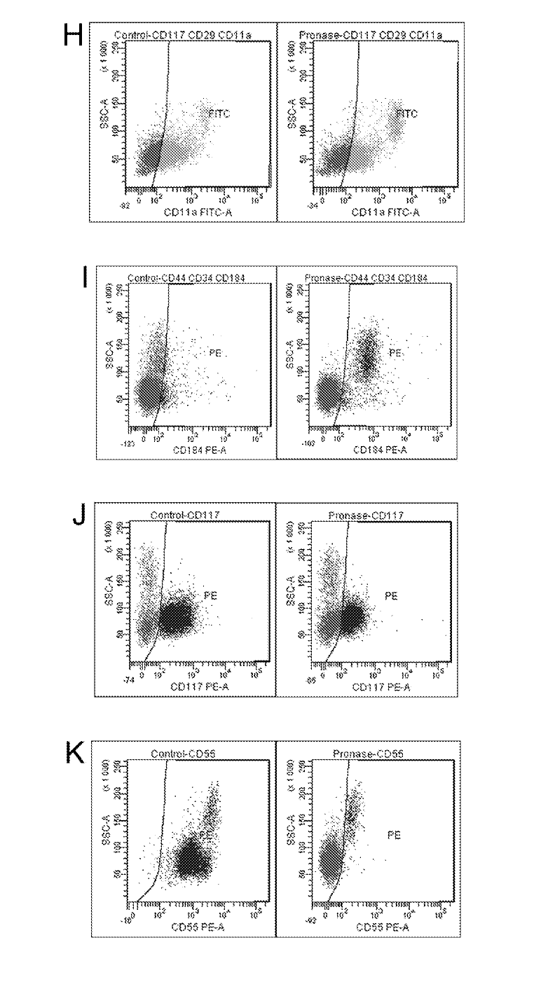

Pronase efficiently cleaved certain protein epitopes from the cell surface, and thus produced changes in the cell surface profile as defined by antibody binding and analysed by flow cytometry (FIGS. 2 and 3). In particular, the bindings of anti-CD44 (H-CAM, hyaluronan receptor), anti-integrin CD49e (ITGα5 or VLA-5), and anti-CD162 (P-selectin ligand 1, PSGL-1) antibodies were essentially completely vanished after the pronase treatment as compared to more than 80-90% of the untreated cells expressing these markers. Furthermore, a clear reduction (down to 10%-20% from the original 85% to 99% in the untreated cells) after the pronase treatment was seen for the CD54 (ICAM-1) and CD55 (decay-accelerating factor, DAF).

In addition, some other reductions in the levels of antibody epitopes were detected as shown in FIG. 2, but they were not as prominent as those mentioned above.

Importantly, pronase did not cleave off all proteins from the cell surface (FIGS. 2 and 3). This is demonstrated by practically unchanged expression of, for example, hematopoietic cell markers CD34, CD133, as well CD90 (Thy-1), and CD45 (Leukocyte Common Antigen). The intact expression of CD49d was of interest, since it has a role in targeting HSC to bone marrow.

### Example 2

### Changes in Cell Surface Epitopes of Cord Blood Mononuclear Cells (MNC) After Pronase Treatment

### Materials and Methods

The isolation of human UCB MNCs was done with Ficoll-Hypaque density gradient (Amersham Biosciences, Piscataway, N.J.) according to the manufacturer's instructions.

Pronase treatment: 1×10⁸ MNC were treated in 10 mL with 0.5% (w/v) pronase (Roche) in PBS without Ca & Mg, pH7.2 +0.25 mM EDTA for 5 min. The control cells were in 0.25 mM EDTA-PBS buffer without the pronase treatment. The reaction was stopped with 30 mL of 0.3% Human Serum Albumin in 2 mM EDTA+PBS. Cells were pelleted with centrifugation 500×g for 5 min. The cell number and viability were determined for all samples after every test by nucleocounter.

### Results

Recoveries for UCB MNC, originally 1×10⁸ cells, after pronase treatment and centrifugation were 1×10⁸ for the control sample and 0.9×10⁸ for pronase-treated sample. The viability was 99.9% for both.

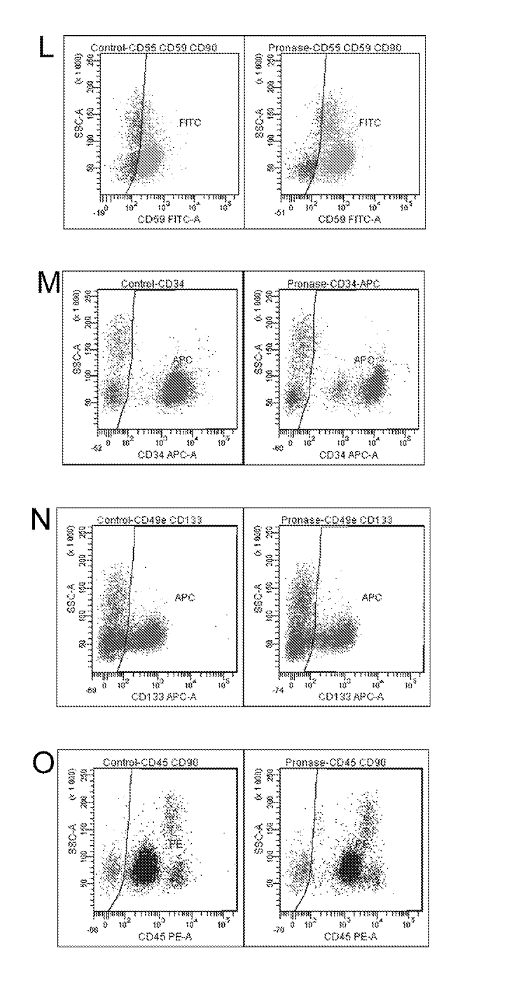

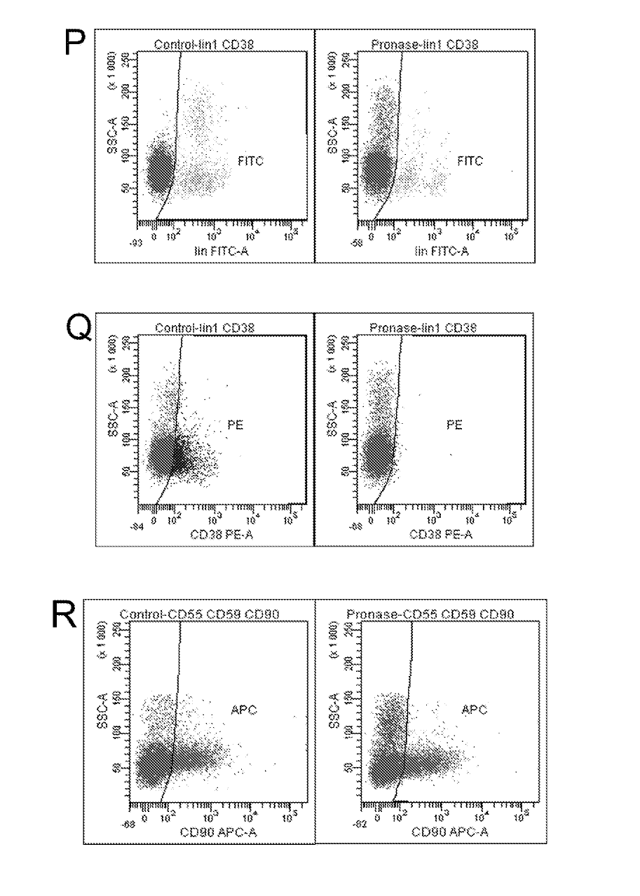

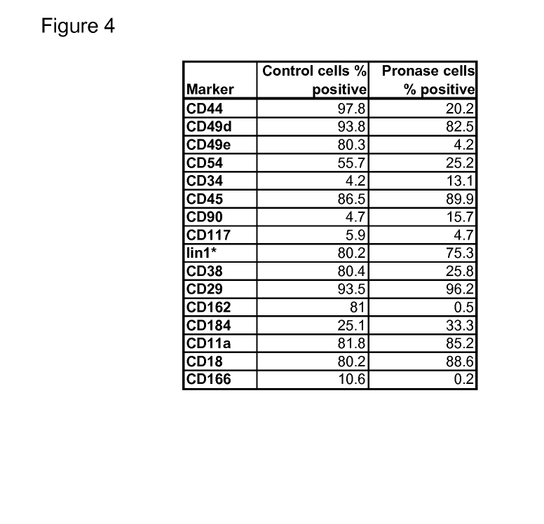

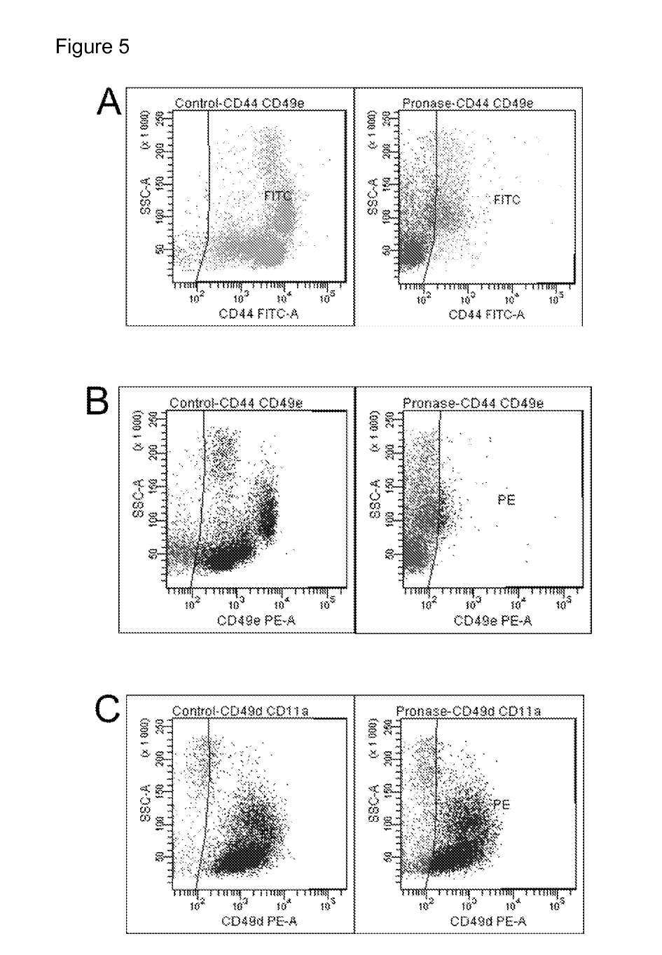

The pronase treatment reduced the expression of the same molecules in UCB MNC as in HSC. Expressions of epitopes for markers CD44 (from 98% to 20%), CD49e (from 80% to 4%) and CD162 (from 81% to 0.5%) were significantly reduced, although the exact figures were not totally identical; in particular for the CD44 marker. Furthermore, the expression levels of epitopes for markers CD49d, CD34, CD45 and CD90 remained essentially the same before and after the pronase treatment (FIGS. 4 and 5). The expression of CD133 was not tested.

### Example 3

### Optimizing the Pronase Treatment

### Materials and Methods

The isolation of human UCB MNCs was done with Ficoll-Hypaque density gradient.

Pronase treatment: For comparison, UCB MNCs were treated with pronase in the “buffer” (=PBS without Ca & Mg, pH 7.2+0.25 mM EDTA) and in the “culture medium” (=αMEM, Gibco +0.5% human serum albumin) with different concentrations and incubation times. In the buffer, the pronase treatments included 0.5%, 0.25% and 0.1% (w/v) pronase for 5 min, 3 min, and 1 min each. In the culture medium the treatments were the same except 0.1% pronase was left out. For each treatment, 1×10⁶ MNCs were used in the total volume of 2 mL. The control cells were in the buffer or medium alone, respectively. The reaction was stopped with 10 ml of 0.3% BSA in 2 mM EDTA+PBS. The cells were pelleted by centrifugation, 300×g for 5 min. The cell number and viability was determined for the strongest treatments (0.5% pronase for 5 min) and controls by nucleocounter. Additionally, aliquots of cells from each treatment were visually examined by microscope and photographed.

Flow cytometry: From each treatment 1×10⁵ cells were labelled with 2 μL of the antibodies against CD44, CD162 and CD54 and their expression levels were analysed essentially as described in example 1.

### Results

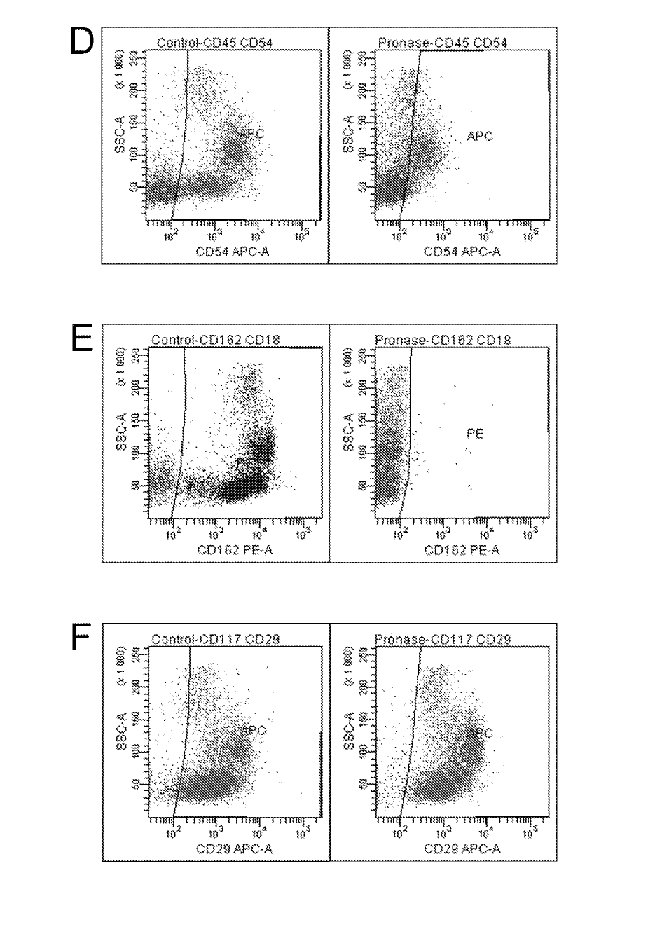

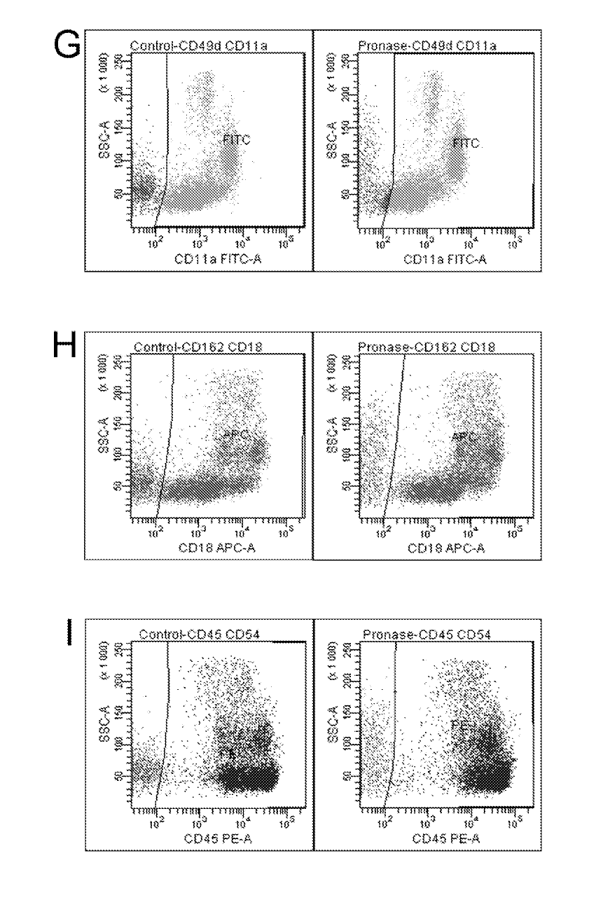

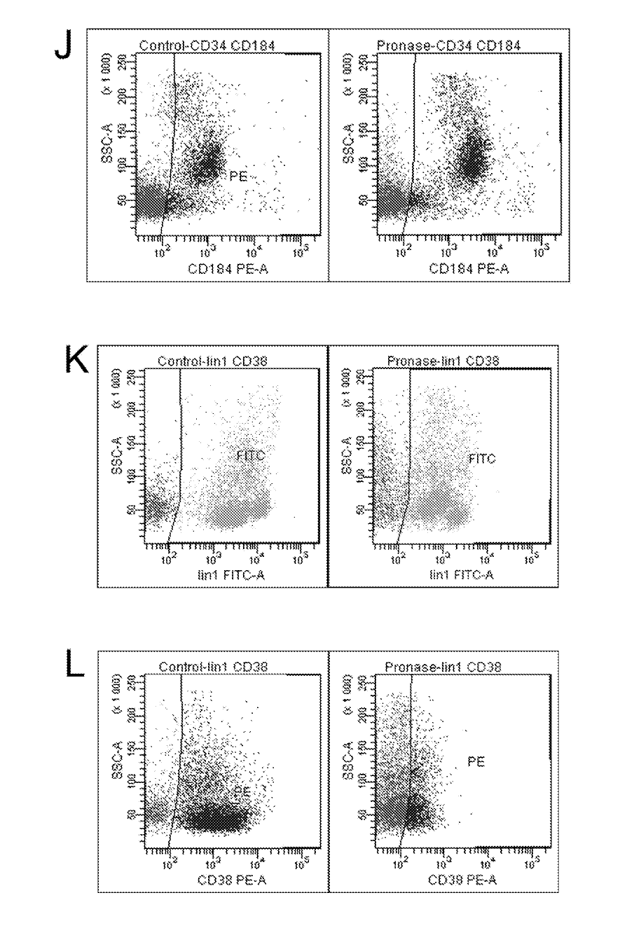

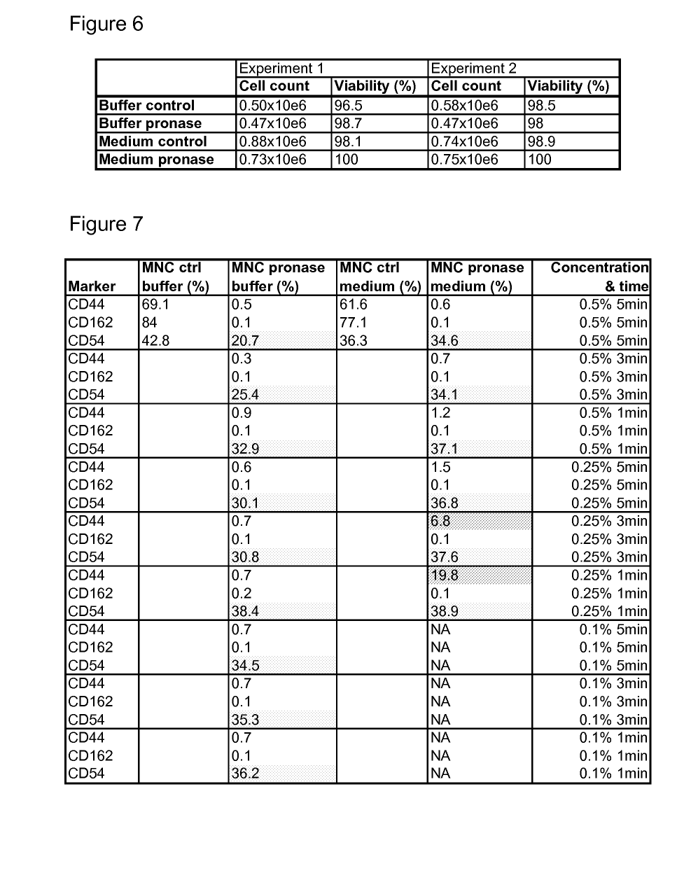

When the samples treated with the strongest (0.5% 5 min) pronase were studied with nucleocounter, it was observed that the recovery of the cells in the buffer was clearly lower than the recovery in the culture medium, both for the control and pronase-treated samples. Originally 1×10⁶ cells were treated and after centrifugation and resuspension there was only about 50% of the cells left in the buffer, while in the culture medium the recovery was about 75% (details not shown). The recovery was slightly higher in the control samples than in the pronase-treated samples (FIG. 6). The viabilities were over 95% in all samples.

The flow cytometry analysis of three cell surface markers CD44, CD54 and CD162 which all were expected to vanish after the pronase treatment, was done in the different treatment conditions. The results showed that in the controls the percentage of cells positive for each marker was higher in the cells that were in the buffer (FIG. 7). When cells were treated in the buffer, there were no clear differences in the CD44 and CD162 expression between the different pronase concentrations and incubation times, i.e. 0.1% pronase was sufficient for cleaving those proteins from the cell surface in one minute. Instead, CD54 was not cleaved off completely from the cell surface even with the strongest pronase treatment (0.5% 5 min), although a dose response could be seen.

In the medium, CD162 was cleaved completely in all different treatments, while CD44 was cleaved in 0.5% pronase in 5 min, 3 min and 1 min and 0.25% pronase in 5 min but not completely in 0.25% in 3 min or 1 min. CD54 was not completely cleaved with pronase when treatment was done in medium or buffer.

### Example 4

### Functionality of Pronase-Treated Cells

### Materials and Methods

To demonstrate that pronase-treated HSC cells remain functional, the CFU (colony forming units) assay was performed with MethoCult H4434 (StemCell Technologies), according to the manufacturer's instructions. Two separate experiments were performed. Briefly, CD34+ selected hematopoietic stem cells were treated with 0.5% (w/v) pronase in the PBS without Ca and Mg, pH 7.2+0.25 mM EDTA for 5 min RT as described in Example 1. Untreated cells were used as a control. The viability and number of cells were determined using nucleocounter. The cells were suspended in the medium containing IMDM +2% (v/v) FBS (StemCell Technologies). In the experiment #1, 5000 cells in 300 uL were transferred to 3 mL of MethoCult. Then 3 times 1.1 mL were plated to 35 mm dish and thus the final amount of cells per dish was approximately 1700. The amount of cells plated was decreased in the experiment #2, due to a high number of colonies in the experiment #1. An equal number of control cells and pronase-treated cells were plated each time. Colonies were scored after 14 days of incubation in 37° C. in duplicate dishes.

For UCB MNC, the CFU assay was done essentially similarly as for the CD34 enriched cells (above), but 10 000 cells were plated per 35 mm dish.

### Results

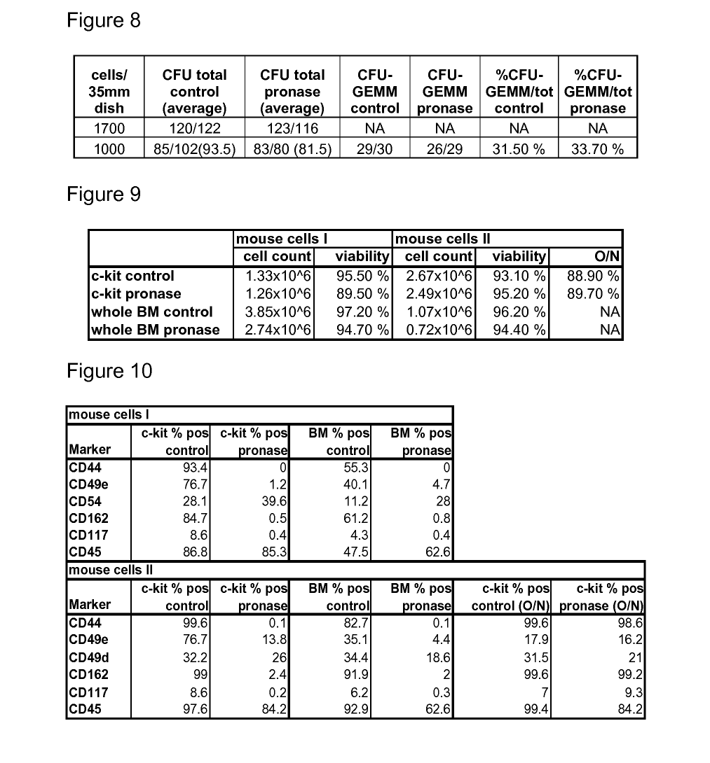

The CFU assay was used to study if pronase-treated HSCs retained their clonogenic capacity. Total CFU (CFU-TOT) number was determined as the sum of granulocyte-erythroid-macrophage-megakaryocyte (CFU-GEMM), granulocyte-macrophage (CFU-GM), erythroid (CFU-E), and burst-forming erythroid (BFU-E) colonies. According to the two independent experiments the same numbers of colonies were formed in the control and pronase-treated UCB HSC. Furthermore, there were equal amount of different colony subtypes; the amount of primitive progenitors CFU-GEMM was determined to be over 30% for both treatments (FIG. 8). Thus, the treatment with pronase did not compromise the functionality of hematopoietic stem cells.

The results with UCB MNC supported the findings obtained using HSC.

### Example 5

### Pronase Treatment of Mouse Cells

### Materials and Methods

The pronase protocol was tested for mouse whole bone marrow (BM) cells and CD117(c-kit)-enriched hematopoietic stem cell precursors that are equivalent to CD34 positive cells in man. The experiment was done twice.

Pronase treatment: The cells were treated with 0.5% (w/v) pronase (Roche) in PBS without Ca & Mg, pH 7.2+0.5 mM EDTA for 5 min. The cells were pelleted by centrifugation (500×g, 5 min) and resuspended in PBS without Ca & Mg pH 7.2+0.5% BSA. Untreated cells were used as control. Cell number and viability was determined for all samples after every test by nucleocounter.

Flow cytometry: In order to ensure the surface modification caused by pronase, aliquots of cells were stained immediately after the pronase treatment with CD44, CD49d, CD49e, CD45, CD162 (PSGL-1) and CD117/c-Kit antibodies. Adequate isotype control antibodies were also used. The labelled cells were run with a FACSAria (BD) flow cytometer and the results were analyzed with the FACSDiva software (BD).

Recovery: Aliquots of pronase-treated CD117(c-kit) enriched cells and controls were left overnight in 37° C. in RPMI +20% FCS and the recovery of the surface proteins was studied using flow cytometry as described above.

CFU assay: To study the functionality of pronase-treated cells, CFU assay was performed as described with Mouse MethoCult H3434 (StemCell Technologies). Mouse whole BM cells and c-kit+ selected hematopoietic cells were plated after pronase treatment at concentrations 2500 c-kit+cells/plate and 25000 whole BM cells/plate in duplicates. Untreated cells were used as a control. Colonies were scored after 8 days.

### Results

Cell viabilities were typically over 90% in control cells and after pronase treatment both for c-kit+ and whole BM cells (FIG. 9). In the overnight recovery samples the viabilities were just below 90%.

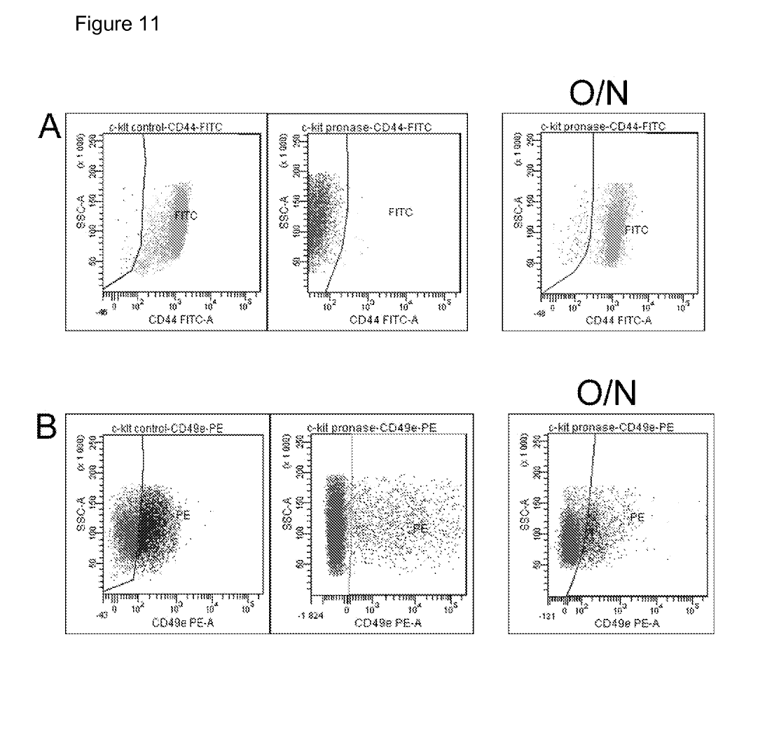

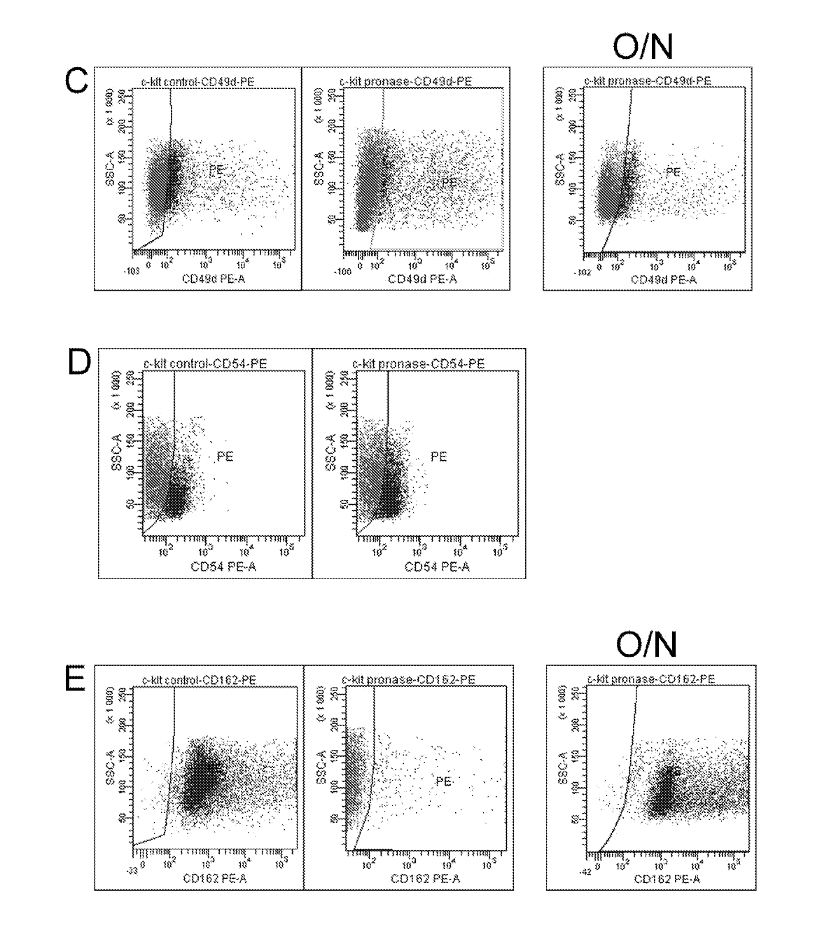

After the pronase treatment, changes were seen in the cell surface protein expression: epitopes for CD44, CD49e, and CD162 were cleaved in both c-kit+ and whole BM cell populations (FIGS. 10 and 11). Hence, the similar pattern was seen as in the human cells.

The CD54 epitope was to certain extend cleaved in the human cells, but no effect was seen in the mouse cells. In fact, there were more CD54 positive cells in the pronase population both in c-kit+ and whole BM cells.

CD45 expression in the mouse cells remained at the similar levels before and after the pronase treatment. The intensity of CD45 epitope expression, however, varied (FIG. 11G).

After overnight incubation in 37° C., proteins that were cleaved by pronase had recovered on the cells surface. This was only verified in the c-kit+ cells (FIGS. 10 and 11A-G).

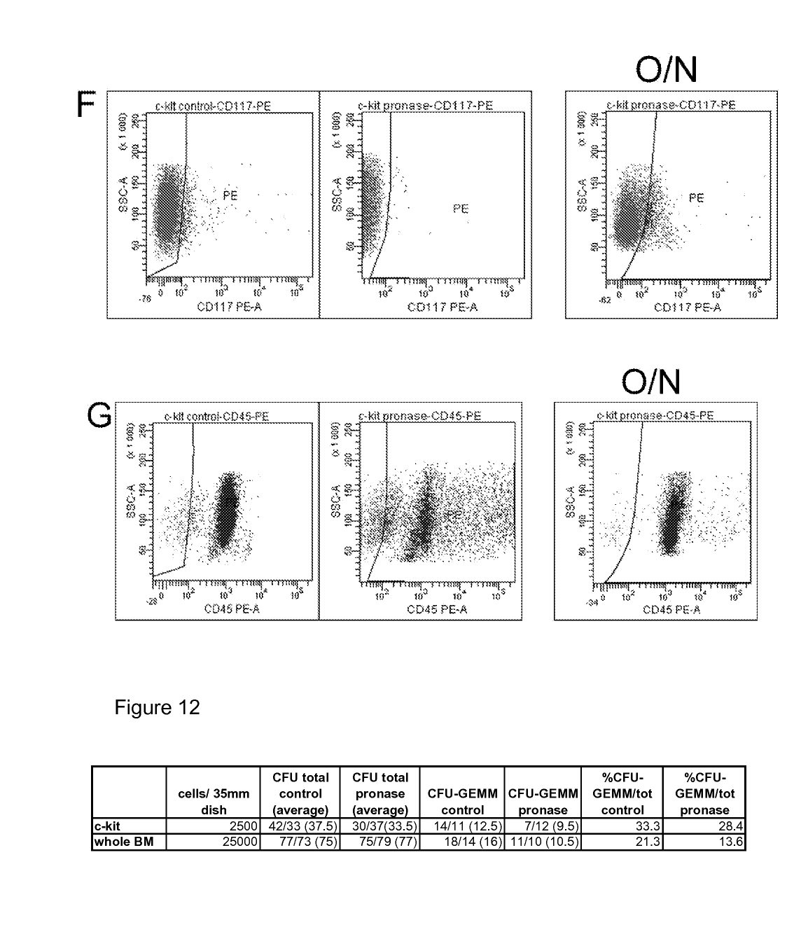

In the CFU assay, the colony forming capacity of pronase-treated c-kit+ cells was not markedly reduced compared to the controls (33.5 vs. 37.5). For the whole BM cells the colony amounts were almost identical (75 vs. 77) (FIG. 12).

### Example 6

### Content of Typical Pronase Product

### Materials and Methods

A sample of pronase enzyme (Roche, #10165921001, lot 70299926) was analysed with mass spectrometry in order to identify the proteins within the sample. The sample was treated overnight in PBS, containing 0.5 mM EDTA and 10% (w/w) modified Trypsin (sequencing grade, Promega Ltd) at 37° C. The protein identification was done both after SDS-PAGE gel separation and using the standard in-liquid reduction, alkylation and digestion procedure.

SDS-PAGE separation was carried out using 12% gel, which was silver-stained as described (Electrophoresis 1997, 18, 349-359). The lower portion of the gel with proteolytically digested protein pieces was selected for in-gel protein digestion. Here, gel pieces were washed and proteins reduced, alkylated and tryptically digested over night as described (Shevchenko et al (2006) In-gel digestion for mass spectrometric characterization of proteins and proteomes. Nature Protoc 1: 2856-2860. 10.1038/nprot.2006.468).

Digested peptides, either from in-liquid or in-gel digest, were analyzed by mass spectrometry. Protein identification was performed with Mascot Server (Matrix Science Ltd., version 2.2.07) against proteins of Streptomyces Griseus in UniProt database (release 2011_{—}03) using search criteria: one potential misscleavage site, variable modifications of carbamidomethyl and propionamide, semitryptic cleavage pattern.

### Results

To characterise typical content of pronase enzyme mixture, an aliquot (Roche, #10165921001, lot 70299926) was analysed with mass spectrometry with two alternative ways. Altogether 16 proteins were found in the direct digestion sample and 38 proteins were found in the gel-digested sample. Seven proteins were found in common between the samples; they were identified with the highest identification scores in both samples. These proteins were: metalloendopeptidase, aminopeptidase, trypsin, putative secreted subtilisin-like serine protease, carboxypeptidase, chain E of structures of product and inhibitor complexes of Streptomyces Griseus protease A, and aminopeptidase S.

1.-15. (canceled)

16. A method of modifying the cell surface of a hematopoietic stem cell (HSC) and/or a population thereof by treating the cell and/or the population thereof with pronase and/or a pronase-like enzyme.

17. The method according to claim 16, wherein the enzyme is pronase.

18. A hematopoietic stem cell (HSC) and/or a population thereof having cell surface protein profile and/or proteoglycan profile, wherein at least one of the proteins selected from the group comprising CD44, CD49e and CD162 is essentially missing.

19. The cell and/or the population thereof according to claim 18, wherein epitopes for CD44, CD49e and CD162 are essentially missing.

20. The cell and/or the population thereof according to claim 18, wherein at least one of the proteins selected from the group comprising proteins CD49d, CD34, CD133 and CD45 is present in the profile.

21. The cell and/or the population thereof according to claim 20, wherein proteins CD49d, CD34, CD133 and CD45 are present in the profile.

22. The cell and/or the population thereof according to claim 18, wherein the level of at least one of the proteins selected from the group comprising CD54 and CD55 is diminished.

23. The cell and/or the population thereof according to claim 18, wherein the profile results from the treatment with pronase or a pronase-like enzyme.

24. A method of assisting the transition of a hematopoietic stem cell (HSC) and/or a population thereof of a graft to the target organ of an individual, wherein the cells are treated with pronase or a pronase-like enzyme and injected to an individual in the need of such engraftment.

25. A method of hindering and/or preventing the transition of a hematopoietic stem cell (HSC) and/or a population thereof of a graft to an organ that is not the actual target one, wherein the cells are treated with pronase or a pronase-like enzyme.

26. The method according to claim 25, wherein the organ that is not the actual target one is lungs and/or liver.

27. A method of modifying and/or altering the distribution behaviour of a hematopoietic stem cell (HSC) and/or a population thereof in a graft, wherein the cells are treated with pronase or a pronase-like enzyme.

28. The method according to claim 24, wherein the enzyme is pronase.
